(19) United States
(12) Patent Application Publication
(10) Pub. No.: US 2003/0129581 A1
(43) Pub. Date: Jul. 10, 2003

(54) Patch-clamping method and apparatus

(76) Inventors: David Geraint Owen, Kent (GB); Andrew John Silverthorne, Cambridgeshire (GB)

Correspondence Address: Pennie & Edmonds, LLP, 3300 Hillview Avenue, Palo Alto, CA, 94304, US

(21) Appl. No.: 10/313,060

(22) Filed: Dec. 6, 2002

Related U.S. Application Data
(63) Continuation-in-part of application No. 09/857,456, filed on Sep. 24, 2001.
(63) Continuation-in-part of application No. PCT/GB01/02490, filed on Jun. 6, 2001.

(30) Foreign Application Priority Data
Jun. 6, 2000 (GB) GB 0013584.8

(51) Int. Cl. C12Q 1/00; C12M 1/34

(52) U.S. Cl. 435/4; 435/287.1

(57) ABSTRACT

A method and apparatus for obtaining a patch-clamp recording from a cell, including providing a microchannel for axial flow of a liquid, providing at least one access port to allow radial access to the interior of the microchannel whereby liquid in the microchannel forms a meniscus at the port and produces an air/liquid interface, providing a patch-clamp pipette having a tip suitable for passing into the access port to form a high-resistance electrical seal between the tip and the cell, passing liquid carrying the cell axially along the microchannel, causing the cell to be carried to the access port, moving the patch-clamp pipette tip and the microchannel relative to each other radially to bring the tip into contact with the air/liquid interface in the access port, and applying suction to the patch-clamp to draw the cell onto the tip to form the seal.

The present application is a continuation-in-part of International Application No. PCT/GB01/02490 filed Jun. 6, 2001 designating the United States, and of U.S. patent application Ser. No. 09/857,456, filed Jun. 5, 2001. Priority is claimed under 35 U.S.C. §120.

### FIELD OF THE INVENTION

The present invention relates to patch-clamping methods and apparatus, and more particularly to semi-automated and automated methods and apparatus for increasing accuracy, speed and efficiency to the patch-clamping procedure.

### BACKGROUND OF THE INVENTION

The basic patch-clamp procedure is now a well understood technique for investigating ion channel activity in cells. Voltage gated ion channels are potential targets for a considerable range of novel treatments in a variety of disease states. The development of the conventional patch-clamp technique has provided a powerful tool for the study of ion channel function and pharmacology in cells. However, while the patch-clamp technique provides a definitive method for the investigation and screening of drugs with potential activity on voltage gated ion channels, the technique is currently highly dependent on the skill of the operator and tends to be very slow for drug screening.

The success of the patch-clamp technique is derived from the ability to form “tight” (i.e. high resistance: giga ohm) electrical seals between an area of the cell membrane (the patch) and the tip of a pipette. The patch-clamp pipette is usually made from glass. The formation of the G-seal is dependent on the profile of the top of the pipette, and is enhanced by the application of suction to the interior of the pipette. The requirements for the formation of the G-seals are well established and the process is usually monitored electrically by display of the current pulse recorded in response to a small voltage step applied throughout seal formation. After formation of a G-seal, the area of membrane under the pipette may be disrupted to obtain whole cell voltage clamp recording mode.

The sequence of events leading to successful G-seal formation and whole cell recording mode using pre-formed patch pipettes is generally as follows:

1. Selection of a suitable cell.

2. The patch pipette is positioned approximately 50 microns above the cell.

3. The pipette is lowered until the cell surface is deformed by the pipette tip.

4. Negative pressure is applied to the interior of the pipette until a G-seal is formed between the pipette tip and the cell membrane.

5. Whole cell recording mode is established by the application of further negative pressure which disrupts the cell membrane in the area under the pipette tip.

Steps two and three are particularly slow and require considerable manual dexterity and a high level of operator skill. Visualization of the cells and the patch pipette requires the use of a high quality microscope and, in order to position the pipette, a high quality three axis micromanipulator with sub-micron resolution in each axis is required.

There is thus a need in the art for apparatus and methods for increasing the rate at which compounds maybe screened for ion channel blocking/agonist activity using the patch clamp technique. Preferably, such methods and apparatus would retain the essential features of the conventional patch-clamp recording system while facilitating automation of the major time-consuming components of the technique.

### SUMMARY OF THE INVENTION

In broadest terms, embodiments of the present invention provide for one or more cell or cells to be suspended in a liquid medium at a liquid/air interface (by virtue of the effect of surface tension at the interface) whereby the cell or cells are accessible at the interface to a microstructure electrode (such as a pipette tip) to which a cell can attach to form an electrical seal, for the purpose of whole cell voltage clamp recording. According to embodiments of the invention, the electrode can be caused to form a high resistance electrical seal with a cell suspended in the liquid at the liquid/air interface without the need to press the cell against a solid support surface.

Any body of liquid or column of liquid, which gives rise to a situation in which a cell or cells are located in the liquid at a liquid/air interface, can be used in the invention. For example cells may be suspended in a column of liquid held by surface tension in a capillary tube. Alternatively cells may be suspended in a droplet of liquid, which droplet may itself be suspended from or supported by a support.

It will readily be appreciate that the patch-clamp technique of the present invention can be operated in “single cell mode”, or could be multiplexed to operate on a matrix of cells with multiple electrodes.

According to one aspect of the invention, interface patching can utilize a patch pipette of conventional type. Cells are supported on a liquid/air interface at one end of a capillary tube (e.g. made of glass, polyethylene or other suitable material). The axis of the patch pipette is in line with the axis of the tube so that the pipette tip can be manipulated into the opening of the tube where the cells are supported at the air/liquid interface. The capillary tube or the patch pipette can be mounted onto a single axis manipulator. Only one manipulator is required and this may be used to move either the patch pipette or the capillary tube.

In an embodiment of the invention, a whole cell recording mode may be established as follows: First, a layer of cells is established at the interface between the extracellular physiological solution (the liquid in which the cells are suspended) and air by dipping the capillary tube into a suspension of cells. The density of cells in the suspension preferably provides a sufficient number of cells to form a layer of cells at the interface. Next, electrical contact with the extracellular solution is established via a non-polarizable electrode (e.g. an Ag/AgCl wire) and the tube is mounted either to a fixed clamp or single axis manipulator. A patch pipette is provided which can be filled with electrolyte solution.

The patch pipette is preferably mounted concentrically with the capillary tube either via a single axis manipulator or fixed clamp (if the capillary tube is to be moved). The pipette filling solution is connected via the nonpolarizable electrode to the headstage of a conventional patch clamp amplifier. The pipette holder allows suction to be applied to the pipette interior. Cell attached patch mode of recording is then established by bringing the pipette tip in contact with the interface by moving the pipette and the capillary tube respectively together along the single mounting axis (e.g. either by moving the pipette towards the tube and interface or vice versa). On entry into the interface the movement of the pipette and capillary tube together is stopped and the pipette current is offset to zero on the patch clamp amplifier. The resistance of the pipette increases when the pipette contacts one of the cells at the air/liquid interface. Suction is then applied to the interior of the pipette and the pipette and capillary tube are moved closer together until the pipette tip is located inside the capillary tube.

Initial seal formation between the pipette tip and the cell may also be assisted by the application of gentle suction during entry of the pipette into the interface. A G-seal is formed between the patch pipette tip and the cell membrane by the application of further suction to the interior of the pipette and monitoring the pipette resistance.

Following the formation of cell attached patch mode, the suction is released, pipette current is offset to zero and a holding voltage applied to the pipette (e.g. −60 mV). A whole cell recording is obtained by the application of further suction to the pipette interior until the whole cell recording mode is established in conventional manner.

According to this embodiment of the invention, it is preferred that the capillary tube be mounted in an upright orientation (i.e. generally vertically) with the air/liquid interface at the downward end of the tube.

As was mentioned above, it is not essential to the general principle of the invention to use a capillary in order to create a column of liquid which gives rise to a liquid/air interface at which cells can be located. Other ways can be envisaged in which the same effect can be achieved. For example, a droplet or “blob” of liquid may be provided on a support surface. The surface has a hole through it and the droplet covers the hole. Surface tension prevents the liquid from the droplet dropping through the hole. Within the droplet cells are suspended. This allows access to the droplet and the cells contained therein by a suitable electrode such as a patch pipette. Means may be provided for flow of other liquids in to and out of a dish or other container of which the support surface with the hole in it forms a wall. Once a cell has been attached to the electrode, other liquids may be introduced into the container either in batch mode or in flow-through mode in order to result in the cell being exposed at its external surface to the surrounding liquid. Clearly in such an arrangement, the original liquid and the remaining unattached cells will tend to be washed away from the area of the electrode/pipette.

For example, in a further alternative embodiment a microchannel may be used to deliver cells in an axial liquid flow. At least one access port is preferably provided in the microchannel to allow radial access from the exterior of the microchannel (air) to the interior of the microchannel (liquid). As such, liquid in the microchannel forms a meniscus at the port and thus produces an air/liquid interface at the port. A patch-clamp pipette having a pipette tip suitable for passing into the access port suitable for forming a high-resistance (giga-ohm) electrical seal between the tip and the cell is also provided. Liquid carries the cell axially along the microchannel, causing the cell to be carried to the access port. The patch-clamp pipette tip and the microchannel are moved relative to each other radially to bring the tip into contact with the air/liquid interface in the access port. Suction is applied to the patch-clamp pipette to draw the cell onto the tip to form the seal and a patch-clamp recording may be made.

In a further preferred embodiment, the cell has been sorted or selected from a heterogeneous source of cells. Preferably, the cell has been sorted and selected using a fluorescence activated cell-sorter (FACS). More preferably, a plurality of cells are carried to the access port singly in a sequential flow.

In a further embodiment of the invention, an apparatus for patch-clamping includes a microchannel capable of carrying an axial flow of a liquid and a patch-clamp pipette having a pipette tip suitable for passing into an access port in the microchannel. Preferably, the microchannel access port allows radial access from the exterior of the microchannel to the interior.

In further preferred embodiments, the cross-section of the microchannel dimension permits only one cell to pass the access port at a time and the microchannel is tubular. Also, the diameter of the tubular microchannel is preferably between about 1 and 2 times the diameter of a cell to be patch-clamped. The microchannel may have more than one access port spaced axially, and preferably there is more than one microchannel.

### BRIEF DESCRIPTION OF THE DRAWINGS

The invention is illustrated by way of example in the accompanying figures in which:

FIG. 1a is a schematic diagram of a capillary tube containing a suspension of cells according to an embodiment of the invention;

FIG. 1b shows the cells having formed a layer at the air/liquid interface at one end of the capillary tube of FIG. 1a;

FIG. 2 shows a general arrangement of a patch-clamp recording equipment with moveable capillary tube according to an embodiment of the invention;

FIG. 3 shows the cell attached to the patch pipette ready for recording mode;

FIG. 4 shows drug/compound addition during interface patch clamp recording in a start position;

FIG. 5 shows drug/compound addition during interface patch clamp recording with an extracellular solution added to dish and dish moved down;

FIG. 6 shows drug/compound addition during interface patch clamp recording with a solution in dish brought into contact with interface region;

FIG. 7 shows drug/compound addition during interface patch clamp recording with the capillary raised above surface of solution in dish;

FIG. 8 is a schematic diagram of a patch-clamping system according to an alternative embodiment of the invention;

FIG. 9 is an enlarged schematic view of a patch-clamp module according to an embodiment of the invention;

FIG. 10 is a schematic diagram of a perfusion flow controller used in preferred embodiments of the invention; and

FIG. 10a is another view of the perfusion flow controller of FIG. 10.

### DETAILED DESCRIPTION OF THE INVENTION

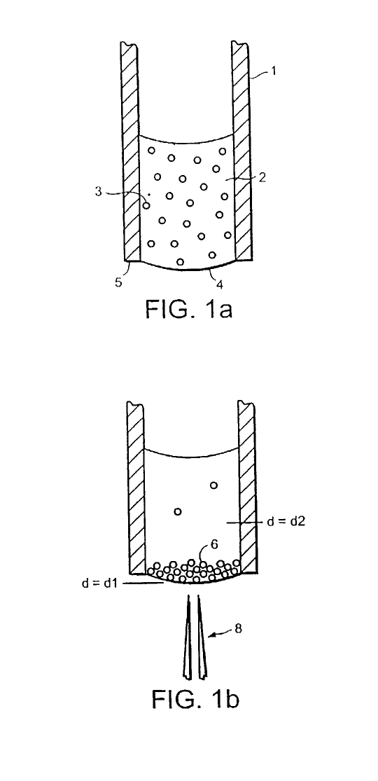

Referring to FIG. 1a, a capillary tube 1 of appropriate size can pick up and hold a liquid sample 2 containing cells 3 in suspension. The sample can be picked up simply by dipping the tube end into a suitable bulk liquid reservoir or the process may be automated. The liquid in the tube forms an air/liquid interface 4 at the tube end 5. The cells are initially distributed throughout the liquid relatively evenly.

As shown in FIG. 1b, with the tube in an upright, generally vertical orientation, the cells tend to sediment and to pack loosely together at the lower end of the tube by the tube end to form a layer 6 several cells deep. It will be appreciated by those skilled in the art that the density and depth of the cell layer can be determined by such factors as the cell concentration in the original suspension, the sedimentation time, the relative density of the cells and the liquid etc. It will also be appreciated that means could be devised to encourage or assist cells to migrate from the liquid towards the air/liquid interface rather than or as well as relying on gravitational sedimentation alone. FIG. 1a also shows the top of a patch pipette 8 pointing upwardly towards the interface.

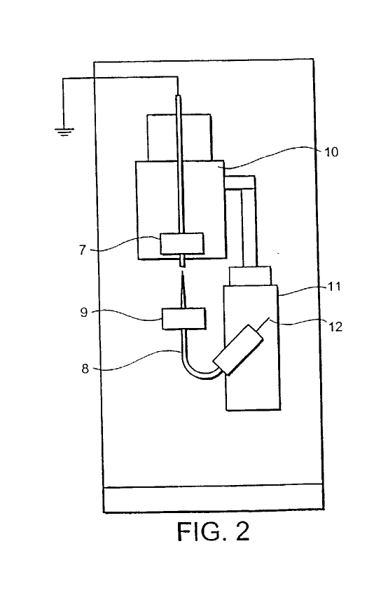

Referring to FIG. 2, an arrangement is shown in which a single axis manipulator is used to move a capillary tube 1 held in a clamp 7 relative to a fixed patch pipette 8 help in a clamp 9. It will be apparent to those skilled in the art that this could be reversed so that the pipette is moved and the tube is fixed. The tube is preferably clamped in a linear bearing sliding block 10 attached to a motorized single axis manipulator 11. The manipulator is controlled preferably by computer in order to allow the motion of the manipulator to be varied by feedback from the patch clamp amplifier. The patch pipette is provided with a connector 12 to a conventional headstage. The system is also provided with a source of variable suction under the control of the patch clamp amplifier/computer.

A version of the apparatus is envisaged in which patch pipettes will be loaded and filled automatically under software control. It is envisaged also that the loading of capillary glass into the apparatus and filling with cell suspension will also be automated.

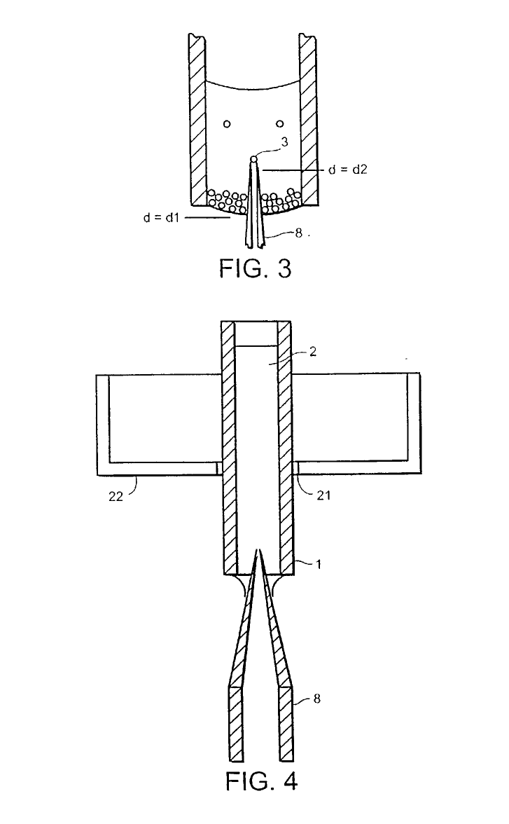

Referring to FIG. 3, a G-sealed cell 3 is shown held on the tip of the patch pipette 8 and positioned within the entrapped liquid volume in the tube. Cell attached patch and whole cell (voltage clamp) recording may then be carried out.

In order to use the invention for screening compound (e.g., for ion channel blocking/agonist activity) the compound of interest needs to be applied to the cell attached to the patch pipette. It will readily be appreciated that this could be achieved in different ways, for example by adding the compound to the extracellular liquid in the capillary tube either before or after G-seal formation. One additional advantage of the invention is that the liquid in the tube could be arranged in layers (e.g., containing different compounds or different concentrations of compounds) and the single axis manipulator could then be used to physically move and position a cell on a pipette tip into a chosen layer (e.g., by moving the G-sealed cell on the tip further up the tube away from the air/liquid interface at one tube end).

A further example of how the effects of compounds may be studied is illustrated in FIGS. 4 to 7. FIG. 4 shows a capillary 1 containing the cell suspension 2 and patch pipette 8 in the recording position for whole cell recording from a cell at the pipette tip. In addition, the capillary tube has been inserted through a hole 21 made in a dish 22 (e.g., 35 mm plastic culture dish or similar). The dish is preferably made of a material with hydrophobic properties and the hole allows the dish to be raised and lowered along the axis of the capillary by means of a micromanipulator 14.

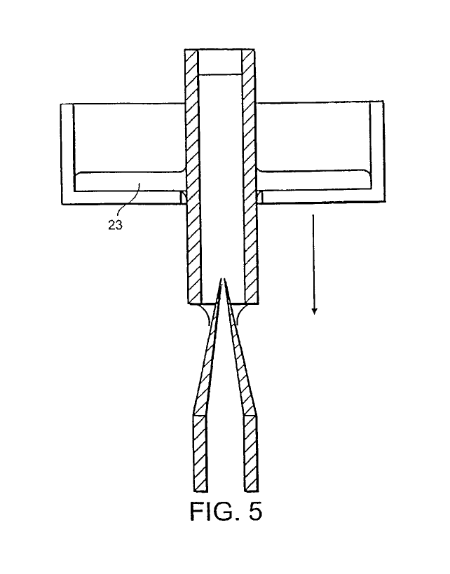

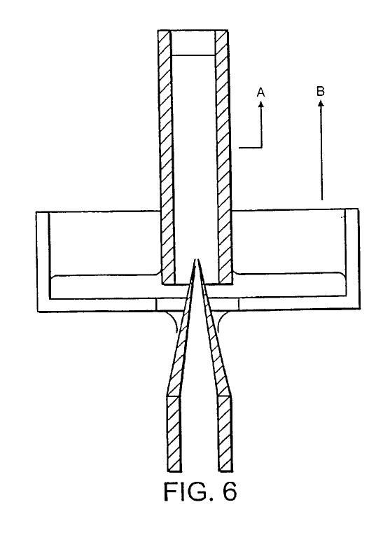

FIG. 5 shows the dish after it has been filled with extracellular physiological solution 23, which may contain the drug to be studied, or the drug may be added at a later stage. If the fluid level in the dish is low, leakage through the hole does not occur because the tendency to leak is counterbalanced by the surface tension of the water and the attraction of the water/solution to the glass capillary. After adding the solution to the dish, it is lowered in the direction of the arrow.

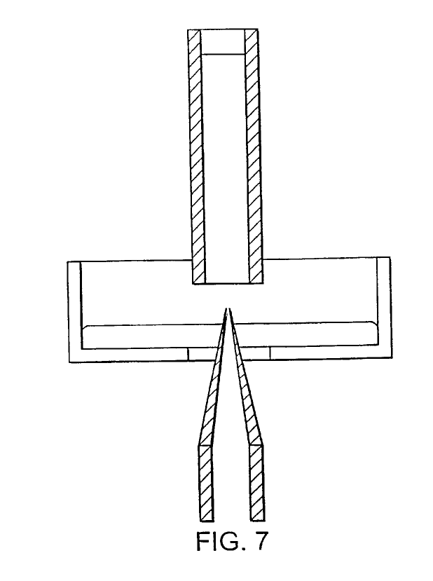

FIG. 6 shows the solution in the dish in contact with the end of the glass capillary and the patch pipette. The dish and the capillary are now raised simultaneously (arrows A and B) in order to position the pipette tip/cell with the layer of liquid in the dish. If drug is present in the dish at this point and the capillary and dish were moved upwards rapidly, this would constitute a rapid application system particularly useful for the study of agonist responses that desensitize.

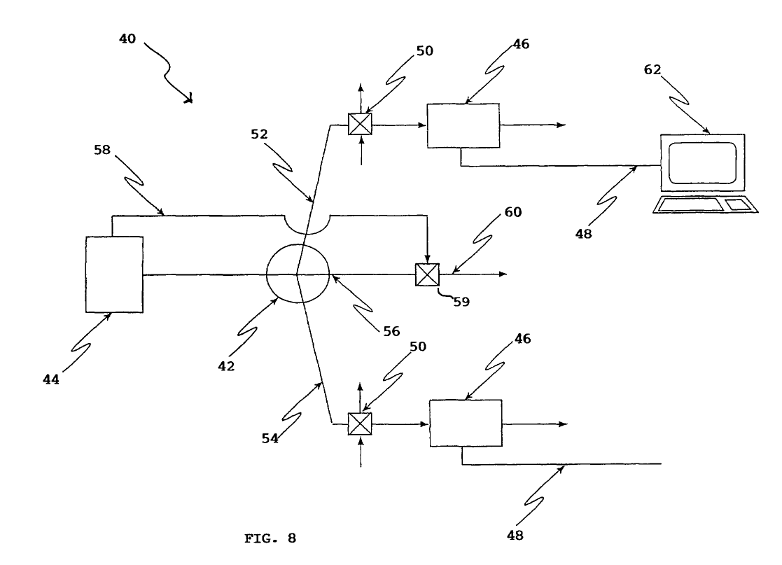

FIG. 7 shows the effect of raising the capillary so that it is not in contact with the liquid in the dish. The pipette tip/cell remains immersed in the external solution layer in the dish. The solution may be exchanged readily by perfusion of the dish and this allows multiple drug additions and dose response curves to be obtained while recording from the one cell.

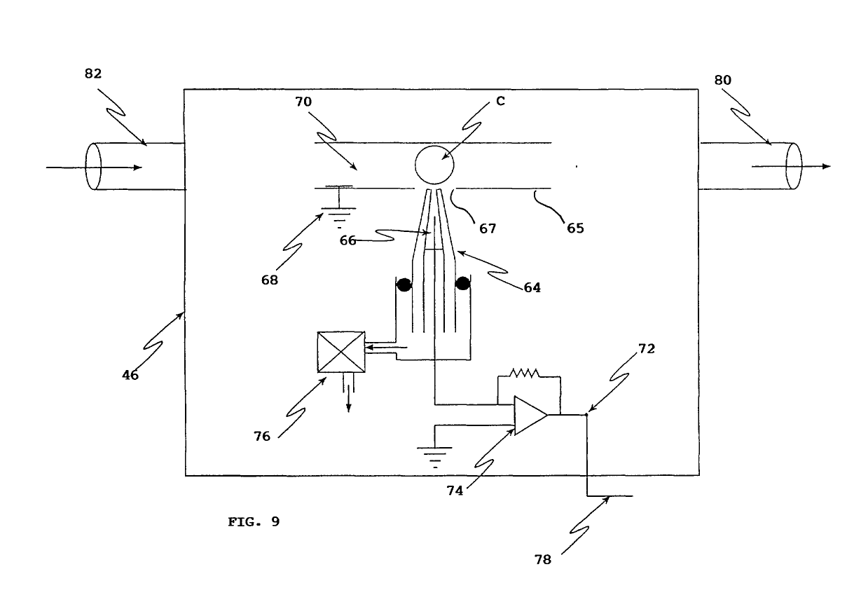

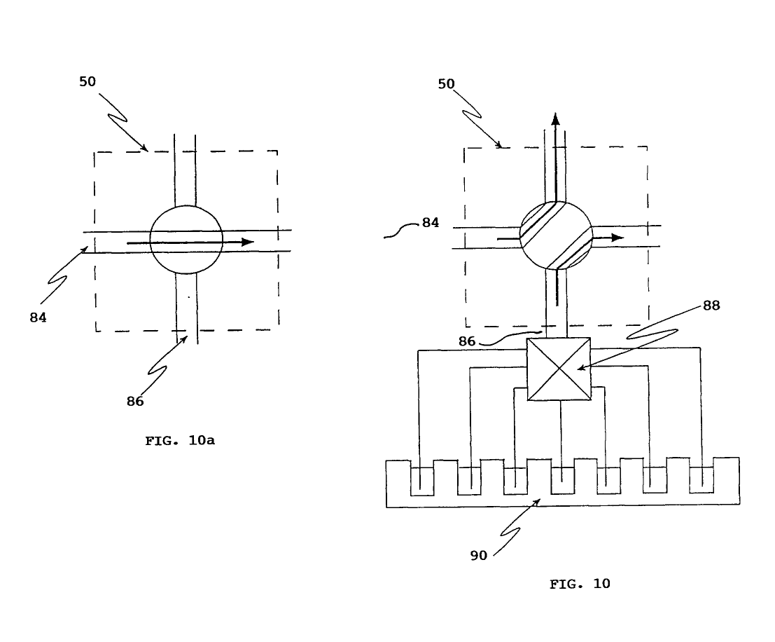

In a further alternative embodiment, as illustrated in FIGS. 8-10A, delivery of cells via a system of microchannels to a patch-clamp pipette provides for a potentially higher degree of automation and accuracy in single-cell patch-clamping. Cells may be pre-sorted using a fluorescence activated cell-sorter (FACS) or other methods of sorting such as immunomagnetic selection. However, the system could also be used without pre-sorting for homogenous cell populations. Delivery of cells and events leading to and including a patch clamp recording are preferably computer controlled as is subsequent drug delivery.

In such embodiments, a patch-pipette accesses cells as they pass an access port in the microchannel. High-resistance electrical seals between pipette and cell (in the order of G or more) are achieved by applying suction to the pipette via a suction controller either on a continuous basis or triggered by the FACS detector which also diverts cells with an appropriate fluorescence signal or light scattering properties along the appropriate microchannel. A minimal system embodiment may comprise a single microchannel with patch-clamp module. Cell suspensions are pumped (e.g., using a peristaltic pump) from a cell incubator through the microchannel. More sophisticated and also higher throughput devices would preferably have a FACS with multi-wavelength capability to permit selection of several cell-types and also multiple patch-clamp modules to permit parallel recording from many cells which may be different or the same in respect to their fluorescence or cell-scattering “signature.”

Referring to FIG. 8, an exemplary patch clamp system 40, according to a preferred embodiment of the invention, optionally includes a FACS 42, cell incubator 44 and patch clamp module 46. Cell incubator 44 provides a source of cells which may be sorted at FACS 42 and provided to one or more patch clamp modules 46 through sorted cell supply lines 52 and 54. Profusion flow controller 50 is preferably disposed in each supply line. Unsorted cells may be recycled to cell incubator 44 from unsorted cell channel 56 through return channel 58. Valve 59 may be provided to select between channel 58 and waste channel 60. Details of suitable FACS are described, for example, in Anderson et al., PNAS 93: 8508-8511 (1996) and Wu et al., Nature Biotechnology 17: 1109-1111 (1999), which are incorporated by reference herein.

In a further preferred embodiment of the invention, patch clamp modules 46 communicate through a control and data acquisition interface 48 with computer workstation 62. Computer workstation 62 preferably includes a computer and standard interface hardware, or other processor and specially designed software and hardware to execute a control logic for automatic control of the system. Exemplary control logics are discussed in more detail U.S. patent application Ser. No. 09/857,456, which is also incorporated by reference herein.

Patch clamp module 46 according to one preferred embodiment of the invention generally includes a patch-clamp pipette 64 positioned under a microchannel 65 defining access port 67 to receive cell C and provide access for the patch-clamp pipette to the cell C. As further illustrated in FIG. 10, patch pipette 64 preferably contains a pipette filling solution or electrolyte 66. Microchannel 65 is provided with a conventional ground connection 68 and contains bathing solution 70. Patch clamp output 72, representative of membrane current, is processed in a conventional manner by patch clamp amplifier 74, which communicates with the control and data acquisition interface through control and data acquisition data line 78. Suction control system 76 provides pressure control for capturing the cell and ensuring the G-seal as described above. Microchannel outflow 80 and inflow 82 provide a suitable path for entry and exit of cells.

As discussed above, patch-clamp pipette 64 is preferably moved upward, generally vertically, in order to contact cell C captured in access port 67. In an alternative preferred embodiment, isolation valves may be provided to isolate a portion of the microchannel surrounding access port 67. In this embodiment, a positive pressure can be applied to force the cell and/or meniscus outward slightly to contact a stationary patch pipette. Further details of such an alternative embodiment are described in pending U.S. patent application Ser. No. 10/239,046, filed Sep. 19, 2002, which is incorporated herein by reference.

Perfusion flow controller 50, shown in FIGS. 10 and 10A, has an inflow from FACS through passage 84 and an inflow from drug application system through passage 86. Manifold and controller 88 switches drug solutions as provided from multi-well plate 90. Collection of drug solutions may be automatically controlled by controller 88, in communication with control system 62.

In one preferred process, according to an embodiment of the invention, cells scanned by the FACS (or not as the case may be) and a cell or cells having an appropriate fluorescence signal is diverted along a microchannel toward a patch-clamp module. Suction is applied to the patch-pipette located in the patch clamp module either at the same time or according to some fixed predetermined interval such that suction occurs as the selected cell passes an access port in the microchannel whereby the cell is drawn to the pipette tip. Alternatively, positive pressure may be used as described above. Seal resistance is monitored automatically and suction controlled by a feedback mechanism under control of a computer. Subsequent steps involved in standard patch-clamping are also preferably determined under software control. Once the desired patch-clamp configuration has been achieved, a perfusion flow controller switches the flow of solution through the microchannel from delivering cells to delivering drug solutions and the experiment is initiated.

Patch-clamp modules may be cascaded such that in the event more than one cell is detected by the FACS, multiple recordings may be made. Excess cells are simply recycled back to the cell incubator.

In the case of a homogeneous source of cells, the FACS front end is not required although it would have the beneficial effect of eliminating debris from the system. In the case where cells come from a mixed background a FACS front end allows selection of even minor components of the overall cell suspension.

The system can be fully automated and because it also recycles cells and solutions, can run for extended periods of time without intervention. A device for supplying conventional glass patch pipettes will be incorporated or alternatively a system for rejuvenating and hence re-using quartz glass pipettes will be used. Data obtained can be automatically downloaded to a server for off-line analysis etc., without interrupting data acquisition.

The foregoing descriptions of specific embodiments of the present invention are presented for purposes of illustration and description. They are not intended to be exhaustive or to limit the invention to the precise forms disclosed. Many modifications and variations will be apparent to persons of ordinary skill in the art in view of the above teachings. The embodiments were chosen and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents.

What is claimed is:

1. A method for obtaining a patch clamp recording from a cell, comprising:
providing a microchannel capable of carrying an axial flow of a liquid;
providing in the microchannel at least one access port to allow radial access from the exterior of the microchannel to the interior of the microchannel, whereby liquid in the microchannel forms a meniscus at the port and produces an air/liquid interface at the port;
providing a patch-clamp pipette having a pipette tip suitable for passing into the access port suitable for forming a high-resistance electrical seal between the tip and the cell;
passing liquid carrying the cell axially along the microchannel, causing the cell to be carried to the access port;
moving the patch-clamp pipette tip and the microchannel relative to each other radially to bring the tip into contact with the air/liquid interface in the access port;
applying suction to the patch-clamp pipette to draw the cell onto the tip to form the seal; and
making a patch-clamp recording.

2. A method according to claim 1, in which the cell has been sorted or selected from a heterogeneous source of cells.

3. A method according to claim 2, in which the cell has been sorted and selected using a fluorescent activated cell-sorter.

4. A method according to claim 1, in which a plurality of cells are carried to the access port singly in a sequential flow.

5. An apparatus for patch clamping, comprising:
a microchannel capable of carrying an axial flow of a liquid; the microchannel having an access port to allow radial access from the exterior of the microchannel to the interior; and
a patch-clamp pipette having a pipette tip suitable for passing into the access port.

6. The apparatus according to claim 5, wherein the cross-sectional microchannel dimension permits only one cell to pass the access port at a time.

7. The apparatus according to claim 5, wherein the microchannel is tubular.

8. The apparatus according to claim 7, wherein the diameter of the tubular microchannel is between 1 and 2 times the diameter of a cell.

9. The apparatus according to claim 5, wherein the microchannel has more than one access port spaced axially.

10. The apparatus according to claim 5, wherein there is more than one microchannel.

11. A method for patch-clamping, comprising:
flowing a cell through a lumen and into communication with a side opening in said lumen;
securing the cell at said side opening;
contacting a patch-clamp pipette with the cell through said opening;
forming a seal between the cell and said pipette suitable for patch-clamping; and
conducting patch-clamping measurements on the cell utilizing said pipette.

12. The method of claim 11, wherein said lumen comprises a microchannel and said side opening forms an access port to an interior of the microchannel, said access port being formed in consideration of the cell size to be patch-clamped to form a meniscus of a carrier fluid in the microchannel at said access port.

13. The method of claim 11, wherein said securing comprises applying a suction at the side opening to fix a cell thereon.

14. The method of claim 11, wherein said contacting comprises moving the cell and pipette relative to one another to contact the pipette with the cell.

15. The method of claim 14, wherein said moving comprises advancing the pipette towards the cell.

16. The method of claim 14, wherein said moving comprises advancing the cell towards the pipette.

17. The method of claim 14, wherein said moving comprises isolating a portion of the lumen containing the cell at the side opening and applying positive pressure in said isolated portion to force a meniscus of fluid out the side opening, forcing the cell contained in the meniscus to contact the pipette.
